(19) United States
(12) Patent Application Publication
(10) Pub. No.: US 2006/0111667 A1
(43) Pub. Date: May 25, 2006

(54) Device and method for controlled expression of gases from medical fluids delivery systems

(75) Inventors: David G. Matsuura, Encinitas, CA (US); Philip J. Simpson, Escondido, CA (US); Walter D. Gillespie, San Diego, CA (US); Davis A. R. Kanbergs, Milton (CA); Taras Worona, Etobicoke (CA)

Correspondence Address: FOLEY & LARDNER LLP, 1530 PAGE MILL ROAD, PALO ALTO, CA, 94304, US

(73) Assignee: Vasogen Ireland Limited

(21) Appl. No.: 10/533,553

(22) PCT Filed: Oct. 28, 2003
(86) PCT No.: PCT/CA03/01645

§ 371 Date: Dec. 6, 2005

Related U.S. Application Data
(60) Provisional application No. 60/421,781, filed on Oct. 29, 2002.

(51) Int. Cl. A61M 31/00

(52) U.S. Cl. 604/93.01

(57) ABSTRACT

Disclosed herein is a syringe assembly for discharging gaseous materials from a syringe, comprising an elongate container with a plunger slidably and sealingly engaged therein to form a fluid material receiving cavity, the container further comprising an outlet for dispensing fluid materials from the cavity; the plunger including transfer means for transferring gas constituents from the cavity to a region outside the cavity.

### REFERENCE TO CO-PENDING APPLICATION

The entire subject matter of U.S. Provisional application Ser. No. 60/421,781 filed Oct. 29, 2002 and entitled DEVICE AND METHOD FOR CONTROLLED EXPRESSION OF GASES FROM MEDICAL FLUIDS DELIVERY SYSTEMS is incorporated by reference. The applicant claims priority benefit under Title 35, United States Code, Section 119(e) of U.S. Provisional application Ser. No. 60/421,781 filed Oct. 29, 2002 and entitled DEVICE AND METHOD FOR CONTROLLED EXPRESSION OF GASES FROM MEDICAL FLUIDS DELIVERY SYSTEMS.

### BACKGROUND OF THE INVENTION

1. Field of the Invention

The present invention relates to medical or other devices for delivering medical or biological liquids by injection or other form such as, for example, syringes and catheters.

2. Description of the Related Art

There has been, in recent years, increasing concern of the safe handling of biological and medical materials. The syringe is a ubiquitous delivery device used in the delivery of such materials and, despite a number of developments over the years, their use poses a significant risk both to the medical professionals using them and the patients receiving treatments.

Prior to the administration (injection) of fluids into the human body, it is clinically necessary to remove most or all of the air from the delivery device. Excessive amounts of air injected into the body can result in an air embolism that can lead to severe complications and even death of the patient. An air embolism is caused by the entry of a bolus of air into the vascular system. The embolism is propelled into the heart, creating an intracardiac air lock at the pulmonic valve and preventing the injection of blood from the right side of the heart.¹
1. Perdue M., Intravenous Complications, Intravenous Therapy, Clinical Principles and Practices, Philadelphia, W. B. Saunders, 1995.

Clinicians have developed techniques to purge unwanted air from a delivery device prior to use. For example, a syringe containing an infusate sample, is prepared by removing the typical protective female LUER cap from the male LUER end of the syringe, thus exposing the latter to the environment. The clinician points the syringe upward and taps it to dislodge bubbles from its inner wall so that they coalesce into a single bubble near the tip. With a gauze pad positioned at the exposed end of the syringe, the clinician then dispenses the syringe plunger to express the bubble.

The clinician must then monitor the progression of the meniscus (formed at the liquid-gas interface), and stop depressing the plunger just as the meniscus reaches the end of the syringe. In the event that the plunger is not stopped in time, a volume of infusate will be displaced from the syringe into the gauze pad. If the gas purge is performed on a syringe with a needle, the latter acts as a nozzle to create a stream of fluid that can, in some cases, shoot several feet For many fluids to be infused, the expelled volume poses little clinical risk to patients or health care staff. However, certain fluids such as blood and chemotherapy agents can pose a serious biohazard contamination risk for patients and staff.

The present invention aims to improve the method by which gas is removed from a syringe or other delivery device.

### SUMMARY OF THE INVENTION

Briefly described, the invention involves a syringe assembly, comprising:
an elongate container with a plunger slidably and sealingly engaged therein to form a cavity to receive fluid materials, the fluid materials including a nongaseous constituent and a gaseous constituent, the container further comprising a first outlet for dispensing fluid materials from the cavity under the action of the plunger;
a gaseous material collection housing having a fluid materials receiving chamber, the housing having a first inlet to couple with the first outlet;
the housing having a second outlet and a second outlet valve portion for controlling the passage of the gaseous constituent from the chamber through the second outlet to a region outside the housing while retaining the non-gaseous constituent within the chamber.

In one embodiment, the assembly further comprises a first inlet valve portion for controlling the passage of the fluid materials through the first inlet The first inlet valve portion includes a valve plate sealingly anchored with the housing adjacent the first inlet. In one example, the valve plate is a slitted disk, a check valve, a duck bill valve, a ball valve, or a combination of two or more thereof. In another example, the first valve portion is a spring-biased “one way” valve.

Preferably, the second outlet valve portion includes a hydrophobic filter media layer sealingly anchored with the housing adjacent the second outlet In one embodiment, the hydrophobic filter media layer includes a substantially wetting membrane or a substantially nonwetting membrane.

In one embodiment, at least a portion of the housing is arranged to view fluid materials accumulating therein In this case, the portion may be at least partially transparent or translucent, or substantially the entire housing may be at least partially transparent or translucent.

In another of its aspects, the invention provides a dispenser device, comprising a chamber to receive fluid materials therein, a movable pressure generating portion to pressure the chamber, the chamber further comprising an outlet for dispensing fluid materials; the pressure generating portion including transfer means for transferring gas constituents from the chamber to a region outside the cavity.

Preferably, the chamber is formed in a syringe barrel and the pressure generating portion is a plunger having at least one passage and a hydrophobic filter layer extending across the passage.

In yet another of its aspects, the present invention provides a syringe assembly for discharging gaseous materials from a syringe, comprising an elongate container with a plunger slidably and sealingly engaged therein to form a fluid material receiving cavity, the container further comprising an outlet for dispensing fluid materials from the cavity; the plunger including transfer means for transferring gas constituents from the cavity to a region outside the cavity the plunger further including at least one passage and a hydrophobic filter layer extending across the passage.

In yet another of its aspects, the invention provides a method for discharging gaseous materials from a medical materials dispenser, comprising the steps of:
filling a medical materials dispenser with fluid materials;
fitting an outlet of the dispenser with an inlet of a collection housing which is arranged to receive fluid materials from the syringe cavity and which has the capability of selectively emitting a gaseous constituent of the material from the housing, and of retaining one or more non-gaseous fluid constituents in the housing;
orienting the dispenser to collect the gaseous constituent adjacent the outlet; and
activating the dispenser so that at least the gaseous constituent exits the outlet and enters the housing wherein the dispensing step may or may not include the emission of the gaseous constituent from the housing while the non-gaseous residual materials are substantially retained therein.

Preferably, the method further comprises the steps of removing the collection housing from the dispenser and actuating the dispenser to administer the fluid materials.

In still another of its aspects, there is provided a process for treating a mammalian patient, which comprises:
extracting an aliquot of the patient's blood with a first medical materials dispenser;
subjecting the aliquot of blood extracorporeally to at least one stressor selected from an oxidative environment, UV radiation and elevated temperature up to about 45° C.;
delivering the so-treated aliquot of blood to a chamber of a second medical materials dispenser;
fitting an outlet of the second medical materials dispenser with an inlet of a residual material collection housing which is arranged to receive residual fluid materials from the chamber and which has the capability of emitting a gaseous component of the material from the housing, and of retaining substantially all non-gaseous fluid materials in the housing,
orienting the second medical materials dispenser to collect, at the outlet, a gaseous constituent in the fluid material within the chamber;
dispensing the medical materials dispenser so that at least the gas constituent exits the outlet and enters the housing, and thereafter;
administering the so-treated aliquot of blood from the second medical materials dispenser to the patient.

In one embodiment, the oxidative environment stressor to which the blood aliquot is a mixture of medical grade oxygen and ozone, with an ozone content from about 0.1-100 μg/ml, the ultraviolet radiation stressor is ultraviolet radiation from UV lamps emitting primarily at wavelengths of 280=n or shorter, for example in the vicinity of 254 nm, mercury line, and the elevated temperature stressor is a temperature in the range from about 38-43° C.

Preferably, the blood aliquot is a volume about 0.1 ml to 400 ml. More preferably, the blood aliquot is 10 cc.

Preferably, the chosen stressor or combination of stressors is applied to the blood aliquot for a period of time from 0.5-60 minutes.

In still another of its aspects, the present invention provides a delivery device, comprising an elongate container with a plunger slidably and sealingly engaged therein to form a fluid material receiving cavity, the container further comprising a gas discharge means for discharging gases from the cavity under the action of the plunger and dispensing means for dispensing fluid materials from the cavity under the action of the plunger.

Preferably, the gas discharge means includes a gaseous material collection housing having an inner gaseous material receiving chamber, the housing having a first inlet to couple with the first outlet; the housing having a second outlet and a second outlet valve portion for the exit of gas from the chamber through the second outlet to a region outside the housing while retaining non-gaseous materials within the chamber.

In one embodiment, the device includes a first inlet valve portion for controlling the passage of the gaseous material through the first inlet. The gas discharge means includes a transfer portion formed on the plunger for transferring a gas constituent from the cavity to a region outside the cavity.

In still another of its aspects, there is provided a gas collection device for a medical fluid delivery system, comprising:
a gaseous material collection housing having an inner gaseous material receiving chamber, the housing having a housing inlet to couple with an outlet of the medical fluid delivery system; and
the housing having a housing outlet and a housing outlet valve portion for controlling the passage of gaseous material from the chamber through the outlet to a region outside the housing while retaining non-gaseous materials within the chamber.

In one embodiment the device further comprises an inlet valve portion for controlling the passage of the gaseous material through the housing inlet The inlet valve portion includes a valve plate sealingly anchored with the housing adjacent the inlet The plate may take a number of forms including a slitted disk. If desired, the valve plate may be spring biased to a closed position to form a unidirectional valve.

In one embodiment, the outlet valve portion includes a hydrophobic filter media layer sealingly anchored with the housing adjacent the second outlet.

The medical fluids delivery system includes a syringe, an IV device, a catheter, or a combination of one or more thereof. In this case, the housing may take the form of a cap and is operable to seal the outlet of the medical fluids delivery system when not in use.

In still another of its aspects, the present invention provides an assembly for discharging gaseous materials from a medical fluid supply device comprising medical fluid dispensing means, the fluid material dispensing means having first outlet means, collection means having a gaseous material receiving means with first inlet means to couple with said first outlet means, second outlet means for emitting gaseous materials from said gaseous material receiving means, second outlet valve means for controlling the emission of gaseous material from said receiving means through said outlet means to a region exterior thereto while retaining non-gaseous materials within the receiving means.

Preferably, the device has a first inlet valve means for controlling the passage of the gaseous material through said first inlet means and the second valve means includes hydrophobic filter means.

The medical fluid dispensing means may include, as an example, a syringe, an IV device, or a catheter, or a combination thereof.

In still another of its aspects, the present invention provides a method for discharging gaseous materials from a medical materials dispenser, comprising the steps of:
filling a medical materials dispenser with fluid materials for later dispensing therefrom;
fitting an outlet of the dispenser with an inlet of a collection housing which is arranged to receive fluid materials from the dispenser and which has the capability of selectively emitting a gaseous constituent of the material from the housing, and of retaining one or more non-gaseous fluid constituents in the housing;
orienting the dispenser to collect the gaseous constituent adjacent the outlet; and
activating the dispenser so that at least the gaseous constituent exits the outlet and enters the housing wherein the dispensing step may or may not include the emission of the gaseous constituent from the housing while the non-gaseous residual materials are substantially retained therein.

In yet another of its aspects, the present invention provides a process for treating a mammalian patient, which comprises:
a step for extracting an aliquot of the patient's blood with a first medical materials dispenser;
a step for subjecting the aliquot of blood extracorporeally to at least one stressor selected from an oxidative environment, UV radiation and elevated temperature up to about 45° C.;
a step for delivering the so-treated aliquot of blood to a chamber of a second medical materials dispenser;
a step for fitting an outlet of the second medical materials dispenser with an inlet of a residual material collection housing which is arranged to receive fluid materials from the chamber and which has the capability of emitting a gaseous component of the material from the housing, and of retaining substantially all non-gaseous fluid materials in the housing,
a step for orienting the second medical materials dispenser to collect, at the outlet, a gaseous constituent in the fluid material within the chamber;
a step for dispensing the medical materials dispenser so that at least the gas constituent exits the outlet and enters the housing, and thereafter;
a step for administering the so-treated aliquot of blood from the second medical materials dispenser to the patient.

In yet another of its aspects, the present invention provides a method for discharging gaseous materials from a medical dispensing device, comprising the steps of:
filling a medical dispensing device cavity with fluid materials for later dispensing therefrom;
fitting an outlet of the medical dispensing device with an inlet of a residual material collection housing which is arranged to receive fluid materials from the cavity and which has the capability of selectively emitting only a gaseous component of the material from the housing, and of retaining other non-gaseous fluid materials in the housing;
orienting the medical dispensing device to collect, adjacent the outlet, a gaseous constituent in the fluid material within the cavity; and
dispensing the medical dispensing device so that at least the gaseous constituent exits the outlet and enters the housing wherein the dispensing step may or may not include the emission of the gas constituent from the housing while substantially all non-gaseous residual materials are retained in the housing.

In yet another of its aspects, the present invention provides dispenser assembly, comprising:
an elongate container with a plunger slidably and sealingly engaged therein to form a cavity to receive fluid materials, the fluid materials including a nongaseous constituent and a gaseous constituent, the container further comprising a first outlet for dispensing fluid materials from the cavity under the action of the plunger;
a gaseous material collection housing having a fluid materials receiving chamber, the housing having a first inlet to couple with the first outlet; and
the housing having a second outlet and a valve assembly for controlling the passage of the gaseous constituent from the chamber through the second outlet to a region outside the housing while retaining the non-gaseous constituent within the chamber; the valve assembly including a first valve portion including an hydrophobic media layer and a normally closed second valve portion spaced from the first valve portion to form an intermediate chamber there between.

### BRIEF DESCRIPTION OF THE DRAWINGS

Several preferred embodiments of the present invention will now be described, by way of example only, with reference to the appended drawings in which:

FIG. 1a is a fragmentary sectional view of a syringe assembly;

FIG. 1b is a plan view of one component of the assembly of FIG. 1;

FIG. 2 is a perspective view of one portion of the assembly shown in FIG. 1;

FIG. 3a is a fragmentary sectional view of another syringe assembly; and

FIG. 3b is a plan view of one component of the assembly of FIG. 3a.

FIG. 4 is a sectional view of another syringe assembly;

FIG. 5 is a perspective view of a component of the syringe assembly of FIG. 4;

FIG. 6 is a magnified fragmentary sectional view of the syringe assembly of FIG. 4;

FIGS. 6a, 6b and 6c are sectional views of alternative configurations for one component of the syringe assembly of FIG. 6;

FIG. 7 is a magnified fragmentary sectional view according to FIG. 6 with the assembly in one operative configuration;

FIG. 8 is a magnified fragmentary sectional view according to FIG. 6 with the assembly in another operative configuration;

FIG. 9 is a magnified fragmentary sectional view according to FIG. 6 with the assembly in still another operative configuration;

FIG. 10 is a magnified fragmentary sectional view according to FIG. 6 with the assembly in still another operative configuration;

FIG. 11 is a magnified fragmentary sectional view according to FIG. 6 with the assembly in still another operative configuration;

FIG. 12 is a fragmentary sectional view of a syringe assembly;

FIG. 12a is a side view of a portion of the assembly of FIG. 12; and

FIG. 12b is a sectional view of a portion of the assembly shown in FIG. 12a.

### DESCRIPTION OF THE PREFERRED EMBODIMENTS

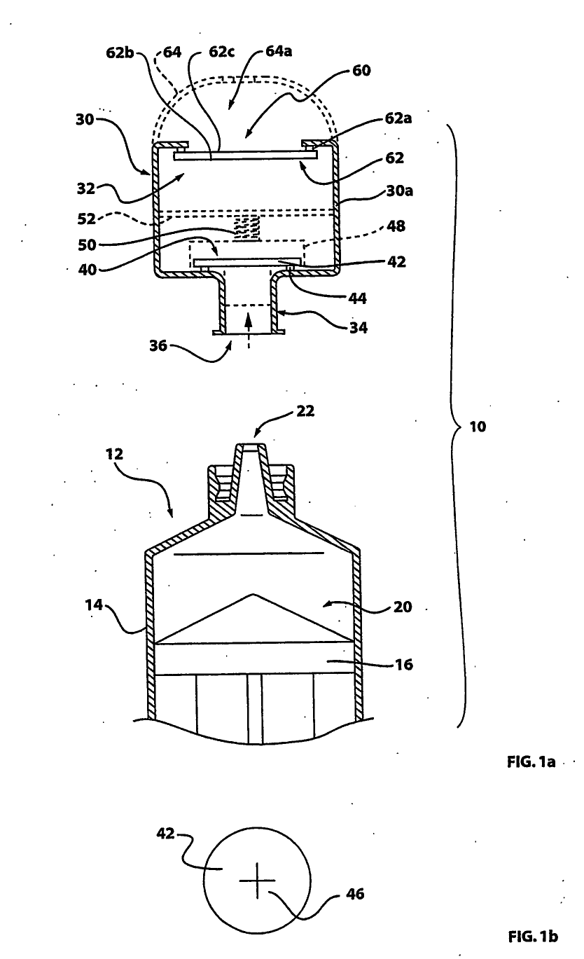

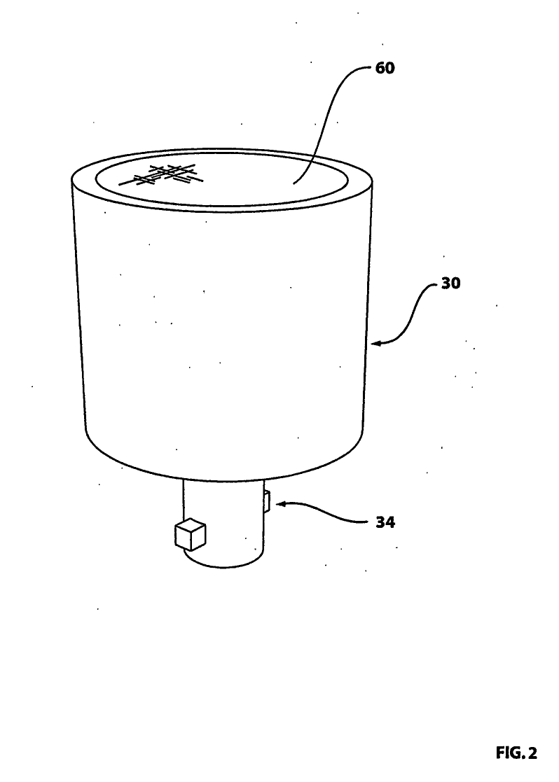

Referring to the FIGS. 1a and 1b, a syringe assembly is shown at 10 for controlling the removal of gaseous materials therefrom In this case, the assembly includes a syringe 12 having an elongate container 14 with a plunger 16 slidably and sealingly engaged therein to form a fluid material receiving cavity 20. The container 14 has a first outlet 22, for attaching therewith a needle (not shown) or other dispensing unit, and for delivering fluid materials from within the cavity 20 to the needle. For example, the first outlet 22 may include a LUER (a trade name) type fitting.

Also provided is a gaseous material collection housing 30 having an outer wall 30a forming an inner material receiving chamber 32 with a necked region 34 to provide a first inlet 36 to couple with the first outlet 22. Located adjacent the necked region is a first valve portion 40 for controlling the passage of the gaseous material through the first inlet and into the chamber 32. In this case, the first valve portion is a valve plate 42 sealingly anchored within the housing adjacent the inlet by way of annular seal 44. As shown in FIG. 1b, the valve plate 42 is provided with a slit 46 to form a bi-directional “two-way” valve. Alternatively, the first valve portion may include a valve member 48 which is spring-biased against the outer wall 30a by way of a spring 50 anchored to a support member 52, as shown in dashed lines in FIG. 1a, to form a unidirectional, or “one-way”, valve. The valve may be formed from a number of materials as will be understood by those of skill in the art to be suitable for medical applications, such as silicone rubber, polyurethane, and the like. The valve may also be provided in other forms, such as ball or “duck bill” type check valves.

The housing 30 is also provided with a second outlet 60 having a second outlet valve portion 62 for selectively permitting the exit of gas from chamber 32 to a region outside the housing while retaining non-gaseous materials within the chamber 32. Preferably, the second valve portion 62 includes a hydrophobic filter media layer sealingly anchored with the housing adjacent the outlet, by way of a seal as shown at 62a In this case, the hydrophobic filter media may also be anti-bacterial with a pore size of 0.2 microns, though other pore sizes and features may also be used. The hydrophobic medial layer has a first surface 62b facing the chamber 32 and a second surface 62c opposite the first surface 62b.

The housing may also include a cowling or external housing portion shown in dashed lines at 64 which is adjacent the second surface 62c, for providing a shield for the exterior of the second valve portion 62. The cowling may have one or more perforations to provide gaseous materials to exit therefrom. The perforations may be in the form of a matrix or other arrangement of relatively small passages or, alternatively, one or more relatively larger passages as shown at 64a.

The syringe assembly 10 may be provided to the user as a package including both the syringe 12 and the material collection housing 30 or be packaged and sold separately.

The syringe assembly 10 may be used in the following manner. First, the syringe 12 is equipped with a needle at the first outlet 22 to penetrate a fluid material source, such as for example a patient, a fluid bearing vial, or a supply channel in a fluid material treatment device such as that disclosed in PCT application serial number PCT/CA00/01078 filed Sep. 15, 2000 entitled APPARATUS AND PROCESS FOR CONDITIONING MAMMALIAN BLOOD (the entire contents of which are incorporated herein by reference). The plunger 16 is then partially retracted, causing the cavity 20 to expand, reducing the pressure therein and consequently drawing fluid materials therein. It is not uncommon, at this stage, to find that the fluid materials now in the cavity include a gaseous constituent which must be removed prior to dispensation of the fluid materials.

The first outlet 22 is then withdrawn from communication with the fluid material source, the needle is removed and the first outlet 22 is then coupled to the first inlet 36 of the material collection housing 30. The syringe 12 is then inverted and tapped or otherwise manipulated to cause the gaseous material constituent to gather at the first outlet 22, it now being at the highest elevation of the cavity 20. The plunger 16 is then depressed into the container 14 to a sufficient degree to cause the gaseous material and an amount, such as for example a residual amount, of other fluid materials from within the cavity 20 to transfer into the first inlet 36 of the housing 30, via the valve 42 and finally into the chamber 32, until such time as the cavity 20 is safely void of all or substantially all gaseous constituents.

The housing 30 may then be removed from the syringe 12 and disposed in the usual manner consistent with the disposal of other medical waste. In this case, the first and second valves prevent emission of any of the residual fluid materials removed from the chamber 32 in the previous step. A needle or other delivery device (such as an IV delivery system) may then be attached to the first outlet 22 and the fluid material administered in a normal fashion by further depressing of the plunger 16.

The housing 30 has the advantage that the cavity 20 can be sized to provide sufficient volume capacity to receive the residual fluid material while the second valve portion 62 is capable of emitting the gaseous constituent to the exterior, if need be, for example, to accommodate additional volume capacity in the chamber 32 for the residual fluid material.

Thus, the assembly 10 is believed to provide a substantial improvement in the safe handling of medical and/or biological materials. The residual material collection housing 30 may, if desired, be configured to function as a cap when not in operation. The second valve portion 62 provides means for gaseous materials to escape the housing 30 and the first valve portion, in this case the first valve plate 42, can be configured to aid, for example, in collapsing bubbles from the fluid materials, such as blood.

The cowling 64 protects the filter media (if exposed on the exterior side of the second valve portion) from damage due to contact with foreign objects. The cowling 64 also prevents the user from direct contact with the filter media, an advantage feature should a minute amount of blood or other materials appear there following use. The housing may also be used with medical dispensing systems other than syringes, such as IV units and the like.

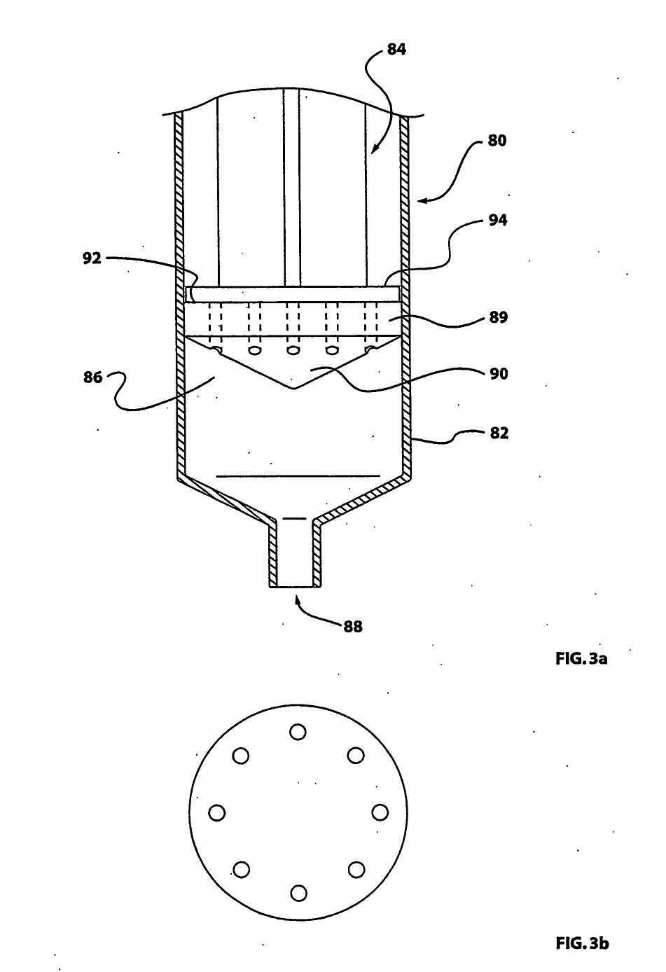

Another syringe assembly is shown at 80 in FIGS. 3a and 3b. In this case, the syringe assembly 80 has an elongate container 82 with a plunger 84 slidably and sealingly engaged therewith to form a fluid material receiving cavity 86.

The container has, at its lower end as viewed in FIG. 3a, an outlet 88 which, similarly to the earlier embodiment, may include a LUER (a trade name) type fitting for attaching a needle or other supply conduit therewith and for permitting the delivery of fluid materials from within the container to the needle.

The syringe assembly 80 has a transfer means for selectively permitting the passage of gas constituents within from the cavity 86 to a region outside the cavity. In this embodiment, the transfer means is integrated in the plunger 84 which has at least one passage, in this case several passages 89, which extend from an inner face 90 of the plunger to an outer face 92 thereof. A hydrophobic filter layer 94 extends across the outer face 92 in order to prevent non-gaseous materials to escape from the cavity passage.

The syringe assembly 80 may be used in the following manner. First, the assembly 80 is equipped with a needle or some other source of fluid material, such as a supply channel in a fluid material treatment device, as described above. The plunger 84 is then partially withdrawn from the container, causing the cavity 86 to expand, reducing the pressure therein and consequently drawing fluid materials therein. In this case, the hydrophobic filter layer 94 may be arranged to prevent or minimize the ingress of gas into the cavity through the plunger 84.

In this case, the plunger is a movable member which, through its movement raises the pressure in the chamber 86. The same function may be applied to other devices having a movable pressure generating member. The filtering action of the hydrophobic filter, via the passages 89, may be replaced by other openings. As a further alternative, the filter 94 may be integrally formed with the pressure generating member, in which case the passages may be incorporated into the filter material, for example to increase the surface area of the filter to improve or increase the rate at which gaseous materials are discharged.

The outlet of the syringe is then fitted with an administration tool such as a needle or other supply conduit and the syringe is oriented to bring any gaseous materials therein against the inner face 90. Dispensing can then continue by depressing the plunger in the normal manner while the gases are simultaneously emitted through the plunger 84 and the hydrophobic filter layer under the pressure exerted by the displacement of the plunger 84.

The assemblies 10 and 80 are particularly adapted for blood and a preferred embodiment of the syringe assembly provides a cavity capable of dispensing 10 cc of blood. Blood presents special handing constraints and the materials used in the assemblies, particularly in the treatment of blood as disclosed in PCT application serial number PCT/CA00/01078 filed Sep. 15, 2000 and entitled APPARATUS AND PROCESS FOR CONDITIONING MAMMALIAN BLOOD, as well as U.S. Pat. No. 6,136,308 to Tremblay filed Oct. 24, 2000 and entitled TREATMENT OF STRESS AND PRECONDITIONING AGAINST STRESS, the entire contents of which are incorporated herein by reference. Accordingly, the components used to make assemblies 10 and 80 are selected from materials which are compatible with blood and the so-formed assemblies are beneficial in that they prevent the blood collected from a patient prior to blood treatment, or blood collected following its treatment, from being contaminated by foreign bodies or materials, or from contaminating foreign bodies or materials, or a clinical environment.

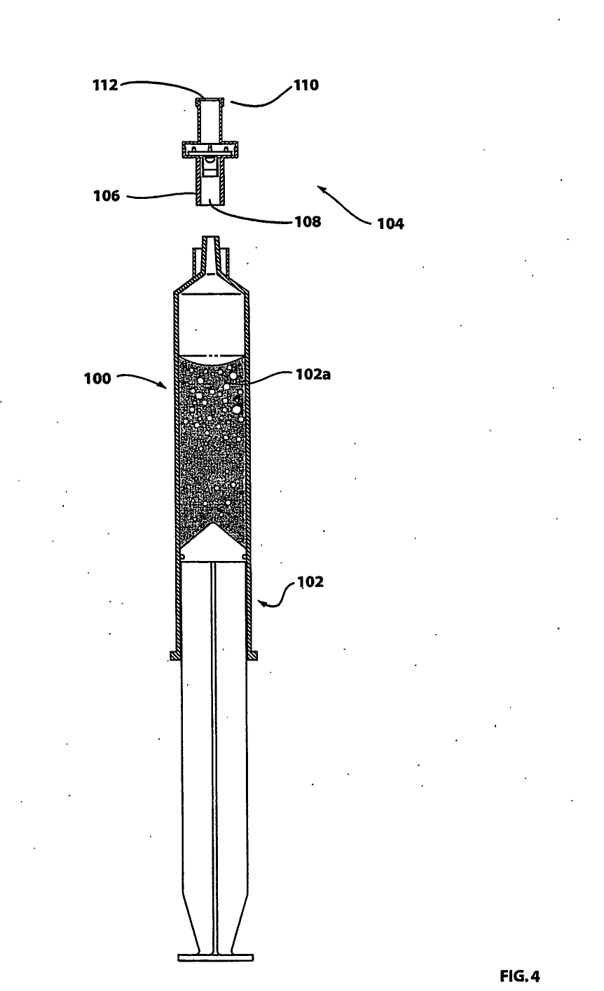

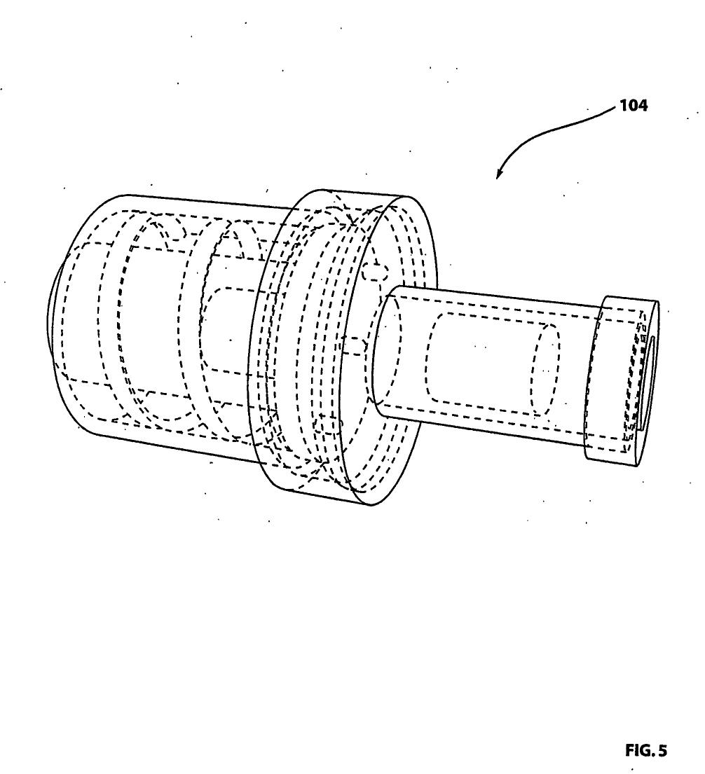

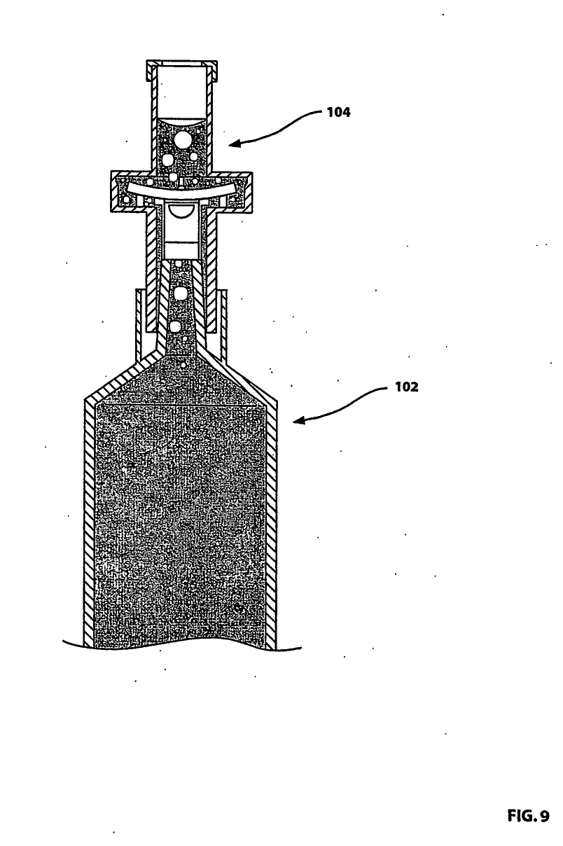

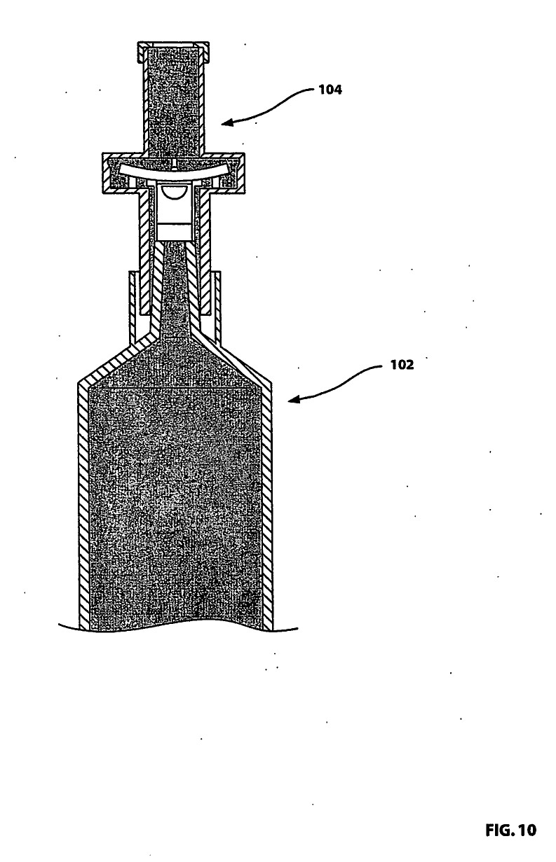

FIGS. 4 to 11 illustrate another syringe assembly 100 having a syringe device 102, having a syringe cavity 102a, and a cap 104. The cap 104 has a body portion 106 which is an adapted LUER activated “reflux valve”, such as those found available from VALUE PLASTICS, INC. under Part Number VPS5401036SN, and manufactured by Burron OEM. In this case, the body portion has one end with a LUER fitting shown at 108 and a distal end 110 to which a hydrophobic filter membrane 112 is ultrasonically welded, thereby covering an aperture at the distal end.

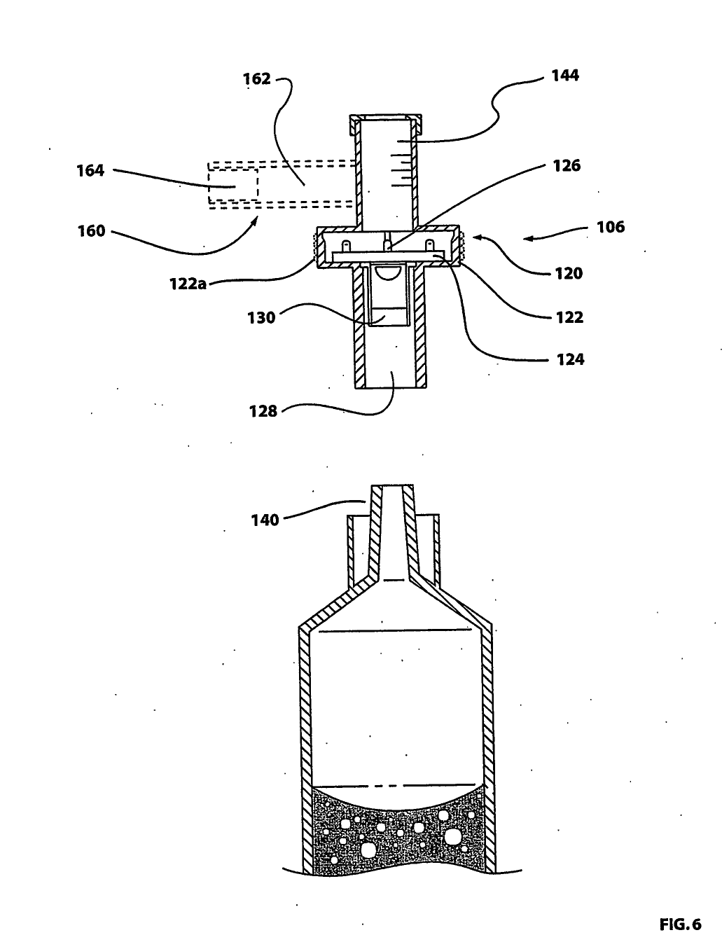

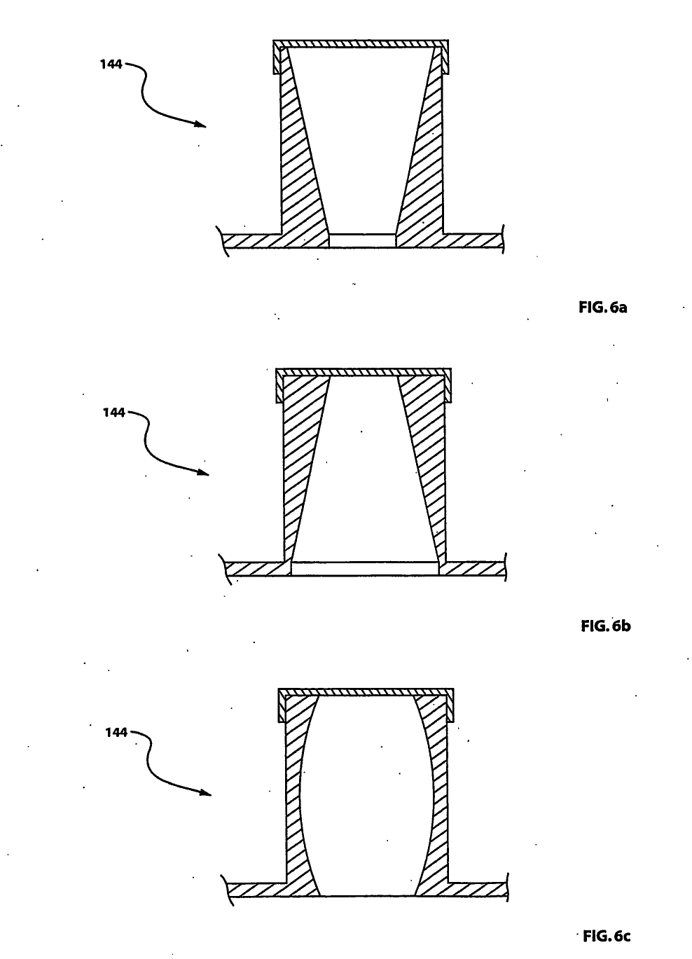

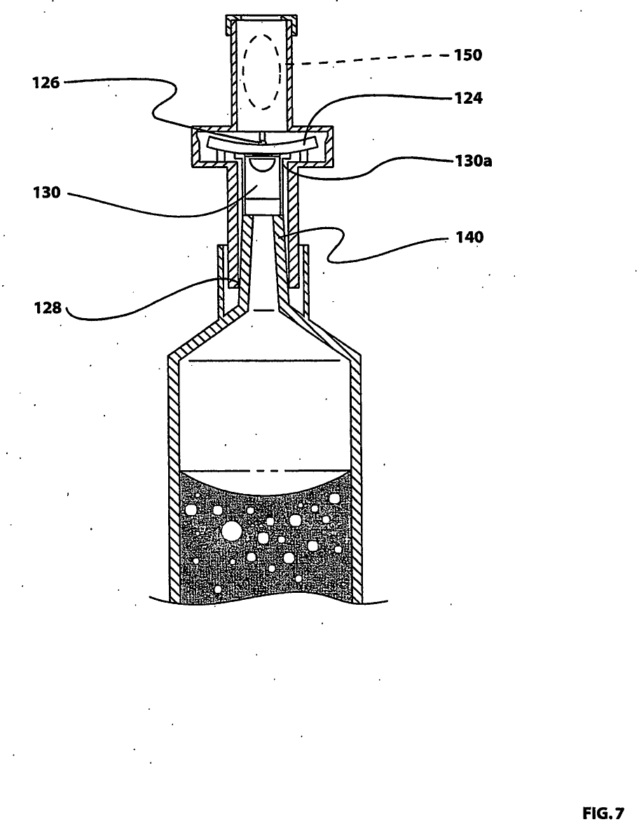

In this case, the hydrophobic filter membrane 112 may be wetting or non-wetting. The latter is preferred and is available from W. R GORE under Part Number 267353885-0, or from other sources such as FILTERTEK of Hebron, IL. This filter media is permeable to gaseous materials and, being hydrophobic, is substantially impermeable to liquids. Moreover, given that the filter is non-wetting, it also repels contact with liquids and therefore mines to a significant extent the occlusion that can otherwise result in a wettable filter. Referring to FIG. 6, the body portion 106 includes a valve 120 having an expanded valve chamber 122 containing a valve plate 124 supported in a closed position by an anchor post 126. The body portion has an inlet passage 128 and an actuator block 130 is slidable within the inlet passage from a lower position as viewed in FIG. 6 to an upper position as viewed in FIG. 7. In the upper position, an upwardly directed peripheral wall 130a on the slide block 130 engages the lower surface of the valve plate 124 and forces it into a tortilla-like orientation against the anchor post 126.

The syringe 102 has a projection 140 which is dimensioned to fit within the inlet passage 128 to engage the block 130 and unseat the valve plate 124. This allows fluid to pass bi-directionally through the LUER fitting and then thereafter to disengage the block 130 thereby releasing it from the valve plate 124, allowing the latter to lie flat once again in its closed position as shown in FIG. 6.

The body also includes an elongate chamber 144 above the valve chamber 122 as viewed in FIG. 6. The elongate chamber is substantially transparent and functions as a sight glass to enable the clinician to view an advancing meniscus during the purging process, as will be described. If desired, the elongate chamber may be provided with indicator markings to record the amount of materials contained in the chamber or, alternatively, be provided with an optically magnified region to aid the clinician to view the meniscus, as shown at 150 in FIG. 7. The clinician may also use the presence of the meniscus in the sight glass element as an early signal to stop depressing the plunger.

The internal form of the elongate chamber 144 providing sight glass lumen may be optimized (smaller, larger, tapered, or otherwise formed in a non-uniform manner) as shown, for example, in FIGS. 6a, 6b and 6c, to provide various levels of feedback to the clinician. For example, the cross section of the lumen may be varied (tapered) along the longitudinal axis of the lumen in order to increase or decrease the meniscus velocity as the sight glass fills with infusate. The external form and walls of the sight glass may also be optimized to create lens like features, to aid the clinician in visualizing the infusate within the sight glass. For example, the sight glass walls may be formed in a manner to provide an optical magnifying function which would minimize infusate loses. Alternatively, marks or gradient lines may be added to the sight glass. Alternatively, mechanical features may be added to the device to provide better grip for handling, such as by way of the formations shown in phantom at 122a in FIG. 6.

The length and diameter of the elongate chamber 144 may be chosen according to the viscosity of the materials, its desired capacity and the degree of control that the clinician wishes to have on the advancing meniscus. For example, the longer the chamber, the longer the period of time the clinician has to gauge when to halt the purging step.

Preferably, the hydrophobic filter media is of the “non-wetting” variety. However, there may be circumstances where a “wetting” type filter is useful, in which case provisions may be made to deal with wetting filters which become occluded with liquids following a gas purging exercise. Once the wetting filter is occluded with liquids, its ability to allow for continued selective gas transfer is reduced. Nonetheless, there are cases where wettable filters may be successfully used, by having an extra air input port in the chamber as generally shown in dashed lines at 160 in FIG. 6 which is in fluid communication with the chamber 144. In this case, the air input port 160 has a passage 162 and a one way valve member shown schematically at 164 which is movable in the passage to permit air into the chamber 144. This extra air input port 160 allows the clinician to draw air into the chamber 144 and, consequently, into the cavity 102a of the syringe 102. This can be useful when the clinician wishes to draw some of the expressed liquid materials from the chamber back into the syringe cavity, where too much liquid was expressed during the first purging attempt In this case, though the wettable filter may be occluded with liquids and its permeability to air severely curtailed as a result The extra air input port 160 thus permits extra air to be drawn into the chamber if needed.

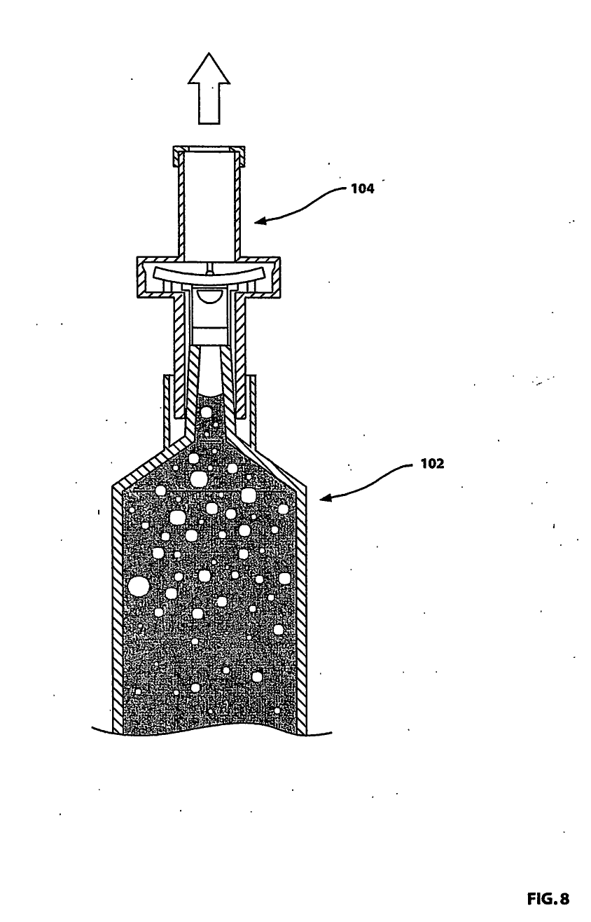

Thus, the cap is capable of gas purging a syringe or other delivery devices commonly used to deliver liquid infusions to the human body, that is while the delivery device remains safely capped, by tilting the distal tip of a syringe upwards to collect the air in close proximity to the filter media, as is shown by FIG. 7. The clinician then depresses the plunger to expel collected gas, as shown successively by FIGS. 8, 9 and 10. As the liquid level reaches the hydrophobic filter media, the plunger force required to generate adequate pressure to pass liquid through the hydrophobic filter media is sufficiently high to signal the clinician to stop depressing the plunger.

If the clinician desires to re-purge the syringe, the non-wetting version of the filter media will allow the clinician to pull back on the plunger in order draw more air into the syringe and re-purge at will. Using this method and device, the clinician can quickly purge the entrapped gas without the concern of fluid escape and consequent contamination. Upon delivery of the infusate, the syringe can be re-capped with the purging cap in order to prevent unintended discharge of residual infusate within the syringe.

The syringe 100 is also useful to “defoam” infusates, such as blood which can in certain circumstances arrive in the syringe cavity with many small bubbles to give the infusate the apparent consistency of foam. The hydrophobic filter media thus enables the clinician, in some cases, to exert relatively high pressures on the blood in the cavity, that are sufficient to collapse the bubbles. This can be done by dispensing sufficient infusate from the cavity into the chamber so that the chamber is full. Continued dispensing will cause the small bubbles to collapse and the gas, neighbouring the hydrophobic filter media, to pass therethrough.

In another aspect of the invention, once the foam buoyantly rises to the top of the syringe, depression of the plunger drives the body of foam into the hydrophobic filter membrane. As compression is applied to the body of foam, the individual bubbles breakdown, thus de-foaming the infusate.

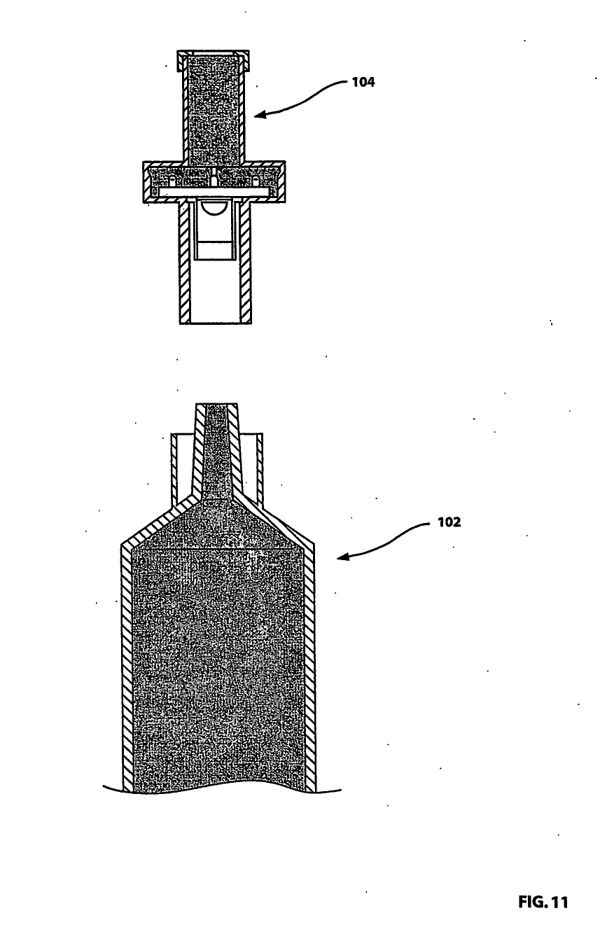

The cap 106 may thus be used as a syringe cap. When gas purging is needed, the assembly may be pointed upward and entrapped gas may be safely expelled without removing the cap. Once the gas has been purged, the syringe may be prepared for administering the medical materials therein by removing the cap as shown in FIG. 11. The cap 106 can thus be removed without spilling its contents, since the LUER activated valve 120 will automatically close when the projection 140 is removed from the passage, thus disengaging the actuator block 130 from the valve plate 124.

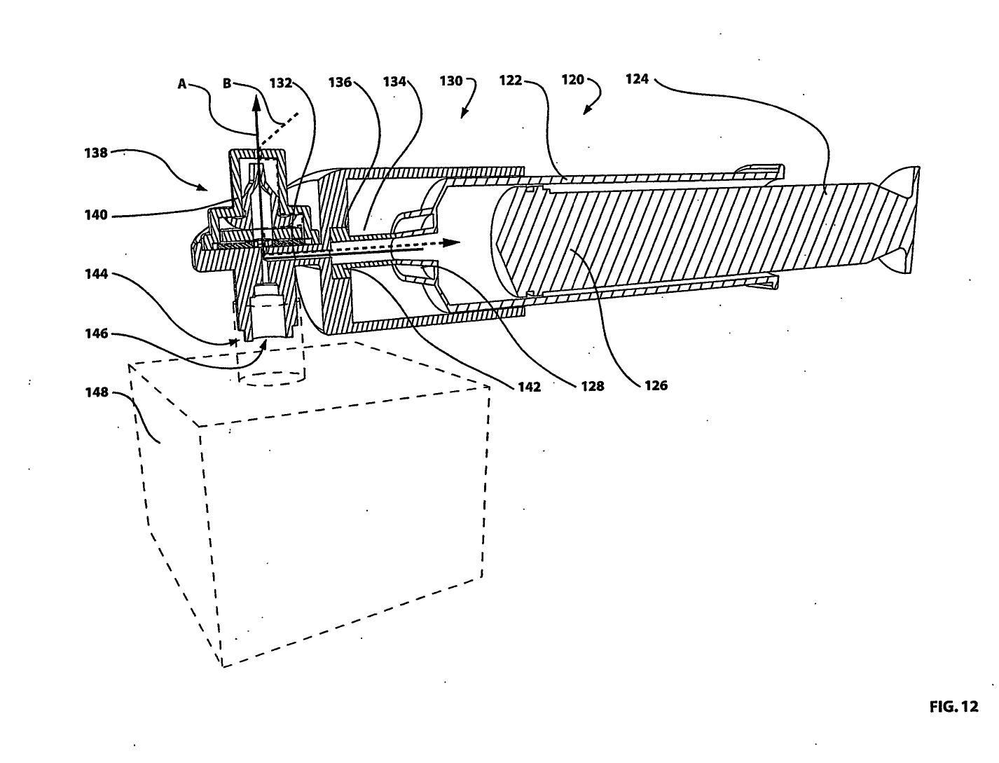

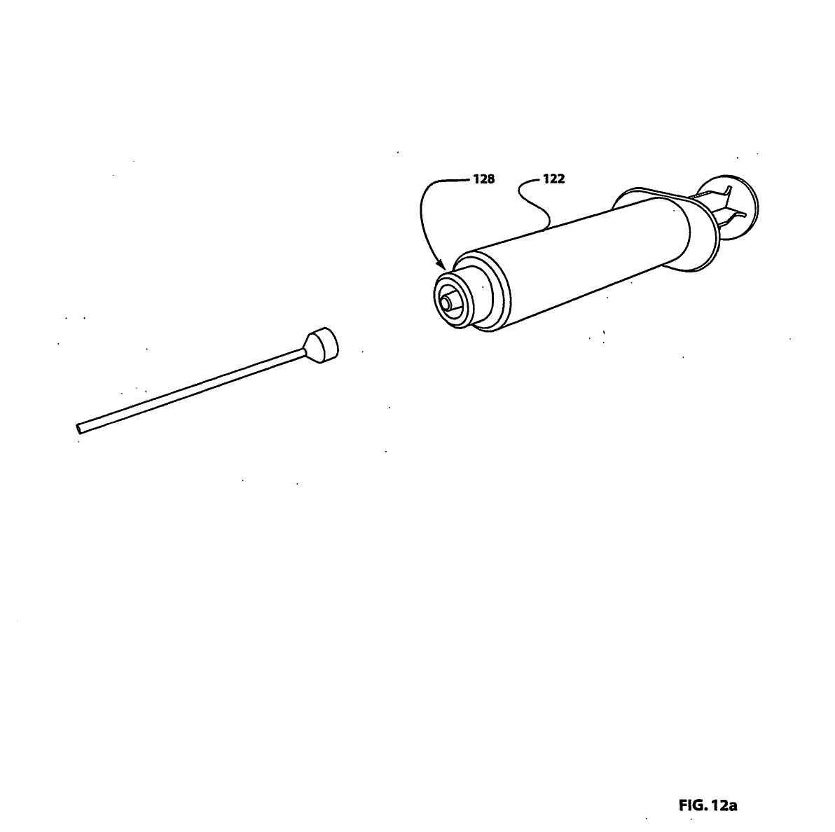

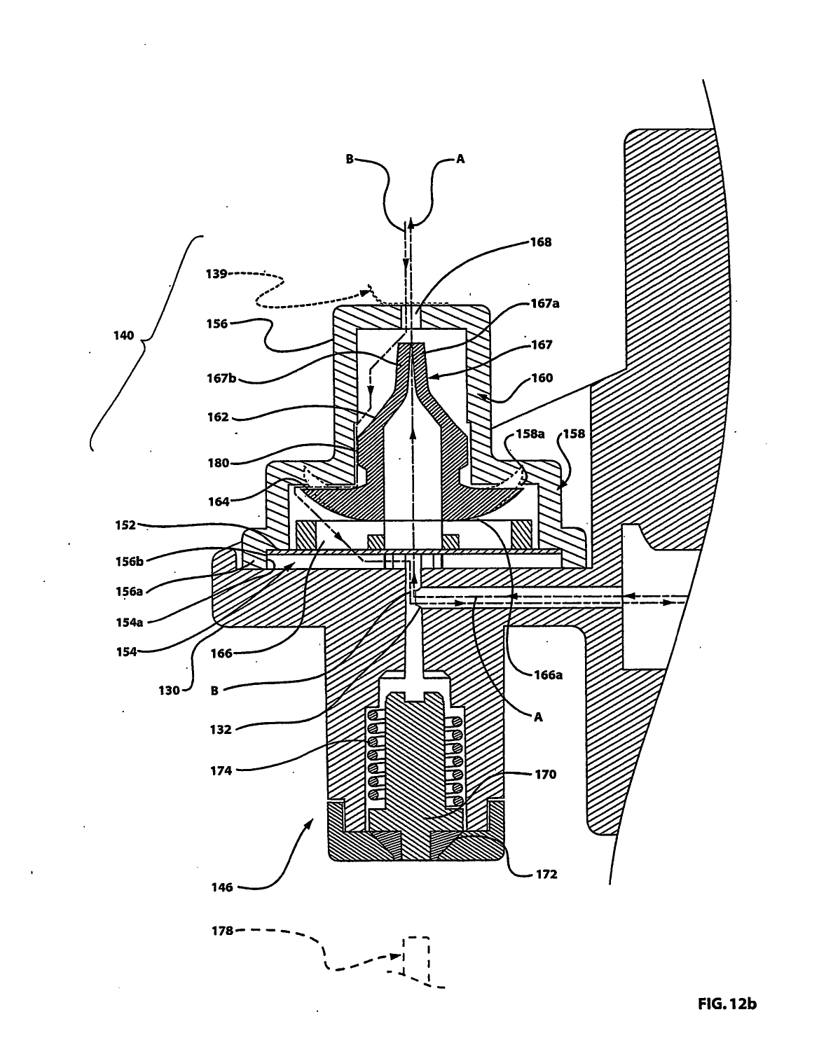

FIG. 12 illustrates a syringe assembly at 120 having an elongate container 122 with a plunger 124 slidably and sealingly engaged therein to form a fluid material receiving cavity 126, the container further comprising a first outlet 128 for dispensing fluid materials from the cavity under the action of the plunger 124. A gaseous material collection housing 130 with a chamber 132 is provided with a first inlet 134 which is coupled with the first outlet 128. Located in the passage is a sealing portion at 136 for establishing a seal between the first outlet 128 and the first inlet 134 minimizing leakage during the transfer of the gaseous material through the first inlet 134. The chamber 132 has a second outlet 138 and a valve portion 140 controls the passage of gaseous material from the chamber 132 through the second outlet 138 to a region outside the housing 130 while retaining non-gaseous materials within the chamber 132.

In this case, as with an earlier embodiment, the chamber 132 provides a passage 142 for the receipt of gaseous materials from the syringe cavity 126. As with an earlier embodiment, the chamber 132 has at least a portion which is relatively narrow and may, in some cases (for example where the housing is transparent) aid in detecting the passage of the meniscus through the chamber 132. As with an earlier embodiment, the chamber is also provided with an additional opening 144. The opening 144 is provided with a valve portion 146 which controls the passage of fluids through the opening 144 which may be used to permit either the entry or exit of fluids or air into or out of the chamber 132. In this particular case, the opening 144 is used for the delivery of a fluid blood sample to the chamber 132 and further to the syringe assembly 120 as will be described. To that end, the opening 144 may be coupled with an external fluids dispensing or delivery device or receptacle, shown schematically at 148 through a suitable fluid coupling therewith.

Meanwhile the syringe assembly 120 is separable from the housing 130 to allow the first outlet 128 to be coupled with the same or other receptacle, collection or delivery device, shown at 12a.

Referring to FIG. 12b, the valve portion 140 includes a hydrophobic media layer 152 as above described, which is held between a seat portion 154 of the housing 130 and a cap member 156 retained thereon. The cap member 156 has a free annular edge region 156a with an inner annular surface 156b whose diameter approximates that of an outer diameter of the complementary outer annular surface 154a on the seat portion 154 to provide a firm fit there between. The cap member 156 has a relatively wide first cap portion 158 and a relatively narrow second cap portion 160. Both portions collectively contain a duck bill valve member 162 therein. The duck bill valve member 162 has an annular flange 164 which is, in its operative position, pressed between an inner sealing surface 158a of the first cap portion 158 and an opposing surface 166a on a spacer member 166, which itself lies adjacent the hydrophobic media layer 152. So positioned, the annular flange 164 is biased away from its natural flared orientation as shown in dashed lines to an urged position against the surface 158a.

As its name suggests, the duck bill valve member 162 has a pair of duck bill shaped valve flaps 167 whose free ends 167a, 167b are normally in contact with one another in a closed position. The valve flaps 167 are operable to separate when the pressure within the chamber exceeds a first pressure level (referred to herein below as the first “cracking pressure”). Similarly, the annular flange 164 is operable to separate from the inner sealing surface 158a when the pressure within the chamber 132 is reduced to a sufficient degree to exceed a second pressure level (referred to herein below as the second “cracking pressure”).

In a first phase of operation, the valve portion 146 remains closed. The syringe assembly 120 is oriented to draw any gaseous constituents in the sample to a region adjacent the syringe outlet 128. Then the plunger 124 is depressed to cause a portion of the fluid material in the cavity 126 to be transferred through the passage 142 into the chamber 132 and a gaseous constituent (if one exists) in the fluid material to pass through the hydrophobic media layer 152 and into the inner region of the duck bill valve member 162. When the sufficient first cracking pressure has been generated by the plunger in the cavity 126 (and hence the chamber 132), the valve flaps 167a, 167b on the duck bill valve member 162 are separated to relieve the pressure, causing the expression of the gaseous constituent downstream of the hydrophobic media layer 152 and within the duck bill valve member 162 (as well as a transfer of other gaseous constituents from the chamber 132 through the hydrophobic media layer 152) and then through an outlet 168 in the cap member 156, following path A as shown in FIG. 12b.

Referring once again to the valve portion 140, the second portion 160 of the cap 156 provides a number of passages 180 for drawing atmospheric air into the chamber 132 and through a momentarily broken seal between the annular member 164 and the surface 158a, along path B, as will be described. This can be useful in cases where the gas purging function may need to be repeated to rid the syringe cavity of lingering gas bubbles, for instance.

Referring now to the valve portion 146, it includes a valve element 170 which is biased against a valve seat 172 under the biasing action of a spring 174 and which is operated by the mechanical interconnection of the second valve portion with a complementary valve actuating member 178 on a mating fitting on the device 148. The valve portion 146 is operable to be opened when the collection housing 130 is interconnected with the device 148 to receive the blood sample therefrom, as can thus occur by drawing the plunger 124 rearwardly in the elongate container 122 to reduce the pressure in the cavity 126 or by pressurizing the device 148 to force the blood sample therefrom.

In this case, when the collection housing 130 is interconnected with the device 148 and the plunger 124 is displaced to draw the blood sample into the cavity 126, it is preferable that the second cracking pressure exceed the pressure differential across the annular flange 164 (that is between the atmospheric pressure on the exterior of the duckbill valve and the relatively low pressure in the chamber) during the drawing of blood into the cavity 126, thereby to avoid air from entering the chamber (and hence the cavity 126) along path B, to result in an added gaseous constituent in the cavity 126 along with the blood sample. If desired, the outlet 138 may be provided with a removable sealing layer 139 (shown in FIG. 12b) to be present when the blood sample is being drawn into the cavity 126 to disable both paths A and B, for example to inhibit air from entering the cavity 126 when the blood sample is being drawn therein. Thereafter, the sealing layer 139 may be removed to permit gas to be expressed through the outlet 138.

The second valve portion 146 is then closed when the collection housing 130 is disconnected from the device 148 thus returning the valve element 170 to its sealed position against the valve seat 172. The sample within the syringe cavity may then be purged of its gaseous constituents in the manner described.

If, during the gas purging process, some stubborn gas bubbles remain in the sample, for instance, the plunger may be retracted to form a lower pressure in the cavity 126 and thus in the chamber 132, until the lower pressure in the chamber 132 exceeds a second cracking pressure limit defined by the annular flange 164 (which will depend, of course, on such things as the dimensions of the duck bill valve member 162, its material specifications and the like) to draw air back into the cavity 126 which may be useful to dislodge the stubborn bubbles, for instance. The second cracking pressure necessary to access path B should be significantly lower than the pressure needed to overcome the spring force on the valve member 170 to ensure that any atmospheric air entering the chamber 134 (and thus the syringe cavity 126) does so via path B, via the annular flange 164 and through the hydrophobic media layer.

Thus, one or more of the embodiments described herein:
1. Minimizes risk of biohazard contamination by providing the ability to purge gas from the delivery device by eliminating unintended discharge of infusate during purging operation.
2. Minimizes risk of biohazard contamination by keeping the delivery device safely capped until administration is required.
3. Minimizes risk of infusate contamination by keeping the delivery device safely capped until administration is required.
4. Minimize risk of male LUER tip contamination, thus patient contamination, of the delivery device by keeping it safely capped until administration is required.
5. Minimizes impact to accepted clinical protocols for gas purging. When used to purge, the device is orientation sensitive emulating current practice. When used as a cap, the device is not orientation sensitive similar to current caps. Its use is relatively simple and intuitive.
6. Provides visual feedback to clinician signalling completion of purge operation.
7. Provides tactile feedback to clinician signalling completion of purge operation by means of a hydrophobic filter membrane that allows the passage of gas but not liquid.
8. Can be used to de-foam infusates without the need for vigorous mechanical input.

While the present invention has been described for what are presently considered the preferred embodiments, the invention is not so limited. To the contrary, the invention is intended to cover various modifications and equivalent arrangements included within the spirit and scope of the appended claims. The scope of the following claims is to be accorded the broadest interpretation so as to encompass all such modifications and equivalent structures and functions.

For example, several embodiments incorporate a lock LUER fitting to couple the delivery device with needles, IV lines or the like. However, other delivery device arrangements may utilize other methods of coupling such as slip LUER fittings, elastomeric seals, snap fittings or threaded fittings.

While several of the above embodiments make use of a plunger to express medical fluids from a cavity into a gas collection housing, it will be understood that the plunger may be replaced by other means for expressing the medical fluid. For example the cavity may be provided in the form of a medical fluids containing bag or other enclosure which may be squeezed, pressed or otherwise manipulated manually or by some other form of pressure generation means to increase pressure within the cavity.

1. A syringe assembly, comprising:
an elongate container with a plunger slidably and sealingly engaged therein to form a cavity to receive fluid materials, the fluid materials including a nongaseous constituent and a gaseous constituent, the container further comprising a first outlet for dispensing fluid materials from the cavity under the action of the plunger;
a gaseous material collection housing having a fluid materials receiving chamber, the housing having a first inlet to couple with the first outlet; and
the housing having a second outlet and a second outlet valve portion for controlling the passage of the gaseous constituent from the chamber through the second outlet to a region outside the housing while retaining the non-gaseous constituent within the chamber.

2. An assembly as defined in claim 1, further comprising a first inlet valve portion for controlling the passage of the fluid materials through the first inlet.

3. An assembly as defined in claim 2, wherein the first valve portion includes a valve plate sealingly anchored with the housing adjacent the first inlet, a slitted disk, a check valve, a duck bill valve, a ball valve, or a combination of two or more thereof.

4. An assembly as defined in claim 1 wherein the second outlet valve portion includes a hydrophobic media layer.

5. An assembly as defined in claim 4 wherein the hydrophobic media layer includes a first surface facing the chamber and an opposite second surface, the second outlet valve portion further including an external housing adjacent the second surface.

6. An assembly as defined in claim 3 wherein the valve plate is spring biased to a closed position to form a unidirectional valve.

7. An assembly as defined in claim 1 wherein the second outlet valve portion includes a hydrophobic filter media layer sealingly anchored with the housing adjacent the second outlet.

8. An assembly as defined in claim 7 wherein the hydrophobic filter media layer includes a substantially wetting membrane or a substantially nonwetting membrane.

9. An assembly as defined in claim 1 wherein at least a portion of the housing is arranged to view fluid materials accumulating therein.

10. An assembly as defined in claim 9 wherein the portion is transparent or translucent.

11. An assembly as defined in claim 10 wherein substantially the entire housing is transparent or translucent.

12. A syringe assembly for discharging gaseous materials from a syringe, comprising an elongate container with a plunger slidably and sealingly engaged therein to form a fluid material receiving cavity, the container further comprising an outlet for dispensing fluid materials from the cavity; the plunger including transfer means for transferring gas constituents from the cavity to a region outside the cavity, the plunger further including at least one passage and a hydrophobic filter layer extending across the passage.

13. A method for discharging gaseous materials from a medical materials dispenser, comprising the steps of:
filling a medical materials dispenser with fluid materials;
fitting an outlet of the dispenser with an inlet of a collection housing which is arranged to receive fluid materials from the syringe cavity and which has the capability of selectively emitting a gaseous constituent of the material from the housing, and of retaining one or more non-gaseous fluid constituents in the housing;
orienting the dispenser to collect the gaseous constituent adjacent the outlet; and
activating the dispenser so that at least the gaseous constituent exits the outlet and enters the housing wherein the dispensing step may include the emission of the gaseous constituent from the housing while the non-gaseous residual materials are substantially retained therein.

14. A method as defined in claim 13 further comprising the steps of removing the collection housing from the dispenser and actuating the dispenser to administer the fluid materials.

15. A method as defined in claim 14 wherein the dispenser includes a includes a syringe, an IV device, a catheter, or a combination of one or more thereof.

16. A process for treating a mammalian patient, which comprises:
extracting an aliquot of the patients blood with a first medical materials dispenser;
subjecting the aliquot of blood extracorporeally to at least one stressor selected from an oxidative environment, UV radiation and elevated temperature up to about 45° C.;
delivering the so-treated aliquot of blood to a chamber of a second medical materials dispenser;
fitting an outlet of the second medical materials dispenser with an inlet of a residual material collection housing which is arranged to receive residual fluid materials from the chamber and which has the capability of emitting a gaseous component of the material from the housing, and of retaining substantially all non-gaseous fluid materials in the housing,
orienting the second medical materials dispenser to collect, at the outlet, a gaseous constituent in the fluid material within the chamber,
dispensing the medical materials dispenser so that at least the gas constituent exits the outlet and enters the housing, and thereafter;
administering the so-treated aliquot of blood from the second medical materials dispenser to the patient.

17. A process as defined in claim 16, wherein the oxidative environment stressor to which the blood aliquot is subjected is a mixture of medical grade oxygen and ozone, with an ozone content from about 0.1-100 μg/ml, the ultraviolet radiation stressor is ultraviolet radiation from UV lamps emitting primarily at wavelengths of 280 nm or shorter, and the elevated temperature stressor is a temperature in the range from about 38-43° C.

18. A process as defined in claim 16 wherein the blood aliquot is of volume of about 0.1 ml to 400 nm.

19. A process according to claim 17 wherein the chosen stressor or combination of stressors is applied to the blood aliquot for a period of time from 0.5-60 minutes.

20. A process as defined in claim 16, wherein the oxidative environment stressor to which the blood aliquot is subjected is a mixture of medical grade oxygen and ozone, with an ozone content from about 0.1-100 μg/ml.

21. A process as defined in claim 16 wherein the ultraviolet radiation stressor is ultraviolet radiation from UV lamps emitting primarily at wavelengths of 280 nm or shorter.

22. A process as defined in claim 16 wherein the elevated temperature stressor is a temperature in the range from about 38-43° C.

23. A delivery device, comprising an elongate container with a plunger slidably and sealingly engaged therein to form a fluid material receiving cavity, the container further comprising a first outlet and a gas discharge means for discharging gases from the cavity under the action of the plunger and dispensing means for dispensing fluid materials from the cavity under the action of the plunger, wherein the gas discharge means includes a transfer portion formed on the plunger for transferring a gas constituent from the cavity to a region outside the cavity.

24. A gas collection device for a medical fluid delivery system, comprising:
a gaseous material collection housing having an inner gaseous material receiving chamber, the housing having a housing inlet to couple with an outlet of the medical fluid delivery system; and
the housing having a housing outlet and a housing outlet valve portion for controlling the passage of gaseous material from the chamber through the outlet to a region outside the housing while retaining non-gaseous materials within the chamber.

25. A device as defined in claim 24 further comprising an inlet valve portion for controlling the passage of the gaseous material through the housing inlet.

26. A device as defined in claim 25, wherein the inlet valve portion includes a valve plate sealingly anchored with the housing adjacent the inlet.

27. A device as defined in claim 26 wherein the valve plate is spring biased to a closed position to form a unidirectional valve.

28. A device as defined in claim 24 wherein the housing outlet valve portion includes a hydrophobic filter media layer sealingly anchored with the housing adjacent the second outlet.

29. A device as defined in claim 24 wherein the medical fluids delivery system includes a syringe, an IV device, a catheter, or a combination of one or more thereof.

30. A device as defined in claim 24 wherein the housing takes the form of a cap and is operable to seal the outlet of the medical fluids delivery system when not in use.

31. An assembly for discharging gaseous materials from a medical fluid supply device comprising medical fluid dispensing means, the fluid material dispensing means having first outlet means, collection means having a gaseous material receiving means with first inlet means to couple with said first outlet means, second outlet means for emitting gaseous materials from said gaseous material receiving means, second outlet valve means for controlling the emission of gaseous material from said receiving means through said outlet means to a region exterior thereto while retaining non-gaseous materials within the receiving means.

32. An assembly as defined in claim 31 further comprising first inlet valve means for controlling the passage of the gaseous material through said first inlet means.

33. An assembly as defined in claim 32 wherein the second outlet valve means includes hydrophobic filter means.

34. An assembly as defined in claim 31 wherein the medical fluid dispensing means includes a syringe, an IV device, or a catheter, or a combination thereof.

35. A process for treating a mammalian patient, which comprises:
a step for extracting an aliquot of the patient's blood with a first medical materials dispenser;
a step for subjecting the aliquot of blood extracorporeally to at least one stressor selected from an oxidative environment, UV radiation and elevated temperature up to about 45° C.;
a step for delivering the so-treated aliquot of blood to a chamber of a second medical materials dispenser;
a step for fitting an outlet of the second medical materials dispenser with an inlet of a residual material collection housing which is arranged to receive fluid materials from the chamber and which has the capability of emitting a gaseous component of the material from the housing, and of retaining substantially all non-gaseous fluid materials in the housing,
a step for orienting the second medical materials dispenser to collect, at the outlet, a gaseous constituent in the fluid material within the chamber;
a step for dispensing the medical materials dispenser so that at least the gas constituent exits the outlet and enters the housing, and thereafter;
a step for administering the so-treated aliquot of blood from the second medical materials dispenser to the patient.

36. A dispenser assembly, comprising:
an elongate container with a plunger slidably and sealingly engaged therein to form a cavity to receive fluid materials, the fluid materials including a nongaseous constituent and a gaseous constituent, the container further comprising a first outlet for dispensing fluid materials from the cavity under the action of the plunger;
a gaseous material collection housing having a fluid materials receiving chamber, the housing having a first inlet to couple with the first outlet; and
the housing having a second outlet and a valve assembly for controlling the passage of the gaseous constituent from the chamber through the second outlet to a region outside the housing while retaining the non-gaseous constituent within the chamber, the valve assembly including a first valve portion including an hydrophobic media layer and a normally closed second valve portion spaced from the first valve portion to form an intermediate chamber therebetween.
